(19) United States
(12) Patent Application Publication
(10) Pub. No.: US 2011/0106223 A1
(43) Pub. Date: May 5, 2011

(54) LIGHT THERAPY SKIN CARE DEVICE

(75) Inventors: Márta Fenyo, Szentendre (HU); Péter Klein, Budapest (HU)

(73) Assignee: POLARIUM HANGULAT ES KOZERZET JAVITO KFT., Budapest (HU)

(21) Appl. No.: 12/994,679

(22) PCT Filed: May 28, 2009
(86) PCT No.: PCT/HU2009/000047

§ 371 Date: Dec. 28, 2010

(30) Foreign Application Priority Data
May 28, 2008 (HU) P0800340

(51) Int. Cl. A61N 5/06

(52) U.S. Cl. 607/88

(57) ABSTRACT

The invention is a light therapy skin care device that includes light sources in a layout that is configured for lighting of human body's given skin surfaces (B) or its parts. Among light sources, there is a UV light emitting and a UV-free light emitting device, the light sources are connected to a controlled driver unit. The light sources are divided in two groups, where in the first group there are (1) UV light emitting light sources, in the second group (2) UV-free light emitting light sources, and at least one part of the light sources of the second group (2) emits polarised light towards the skin surface (B). The controlled driver unit is configured to control light emission of the light sources of the first (1) and second (2) groups and/or their proportion.

The invention is a light therapy skin care device that includes light sources in a layout that is configured for lighting of human body's given skin surfaces or its parts. Among light sources, there is a UV light emitting and a UV-free light emitting device, the light sources are connected to a controlled driver unit. In the present approach, under skin care device we understand a device that has an effect on skin or on the given entire organism or on both by lighting through skin.

Light therapy devices for cosmetic and skin care purposes that emit light-rays of different wave-length using their advantageous properties are known. The most common is skin irradiation by UV light that results in browning via pigments and other favourable effects. It has the danger that in case of increased usage or sensible skin types it may cause harmful side effects even at smaller irradiation rates. To prevent this, long-time, gradual series of treatment are used.

Moreover, advantageous effect of polarised light both on human organism and directly on skin is known. Such are known, for example from the WO 2006/005976 patent description that describes a procedure and a device for application of polarised light.

Aim of our present invention is to work out a solution that will provide opportunity for application of different treatment modes observing various skin types and at the same time it combines advantageous effects of UV light and polarised light.

Our invention is based on recognition that by integration of different types of light sources into a single devise we can provide optimal effects according to the above aims in a single treatment.

In accordance with the most common design of the invention, in the device according to the introductory paragraph, light sources are divided in two groups, where in the first group there are UV light emitting light sources, in the second group UV-free light emitting light sources, and at least one part of light sources in the second group emits polarised light towards skin surface, further the controlled driver unit is configured for controlling of light emission intensity and/or their proportion.

Further we describe figures that illustrate the invention.

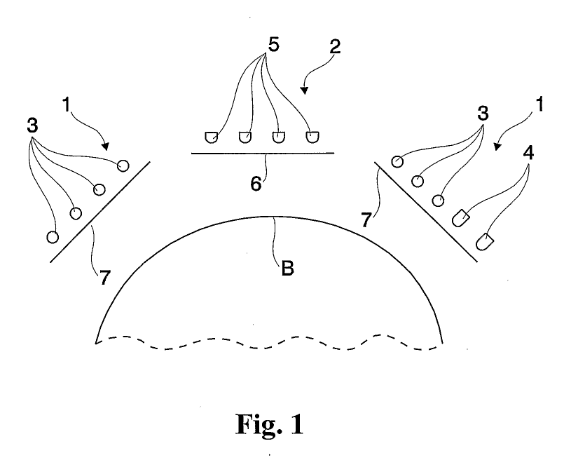

FIG. 1 is a cross section draft about irradiation of skin surface.

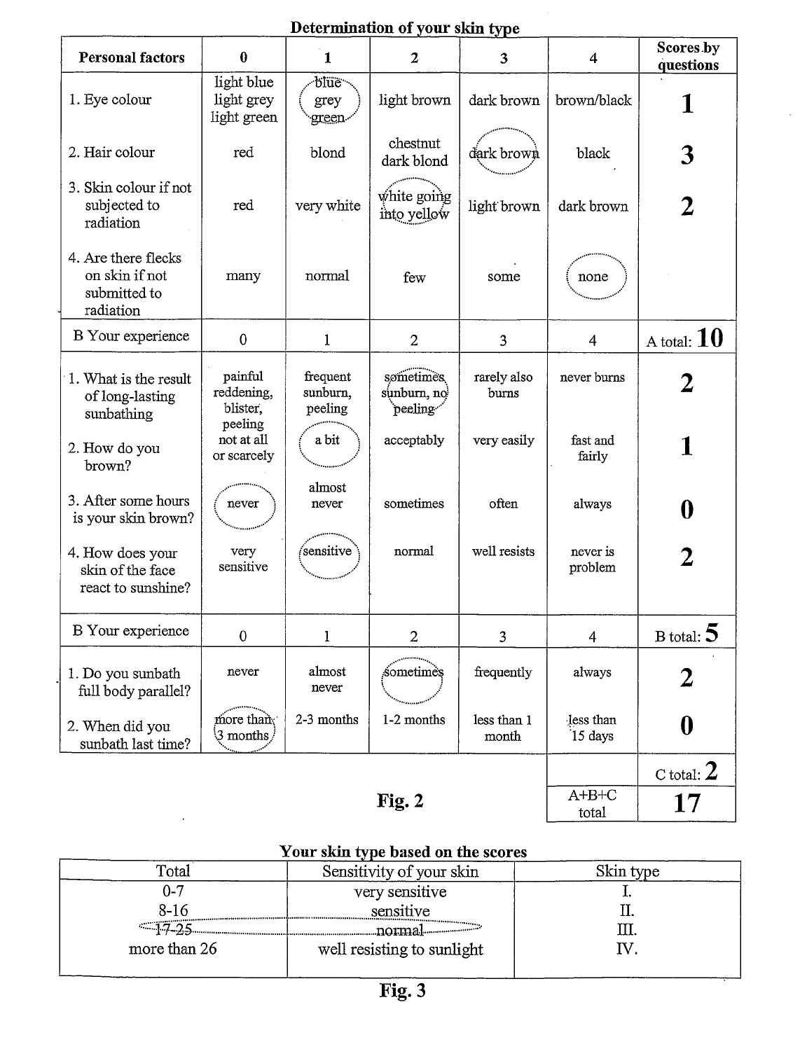

FIG. 2 is an example for a query used for determination of skin type.

On FIG. 1 we illustrated a part B of a person's body's skin surface so that it is irradiated by lighting units included in various groups arranged in one device. According to this, light sources are divided in two groups—in the first group 1 UV light emitting light sources, while in the second group 2 UV-free light emitting LED-s 5 are placed. Light sources can be solarium tubes 3 or UV LED-s. These can also be combined in mixed application, e.g. as it is shown at the right part on the FIG. 1 of the first group 1.

We can summarise that among light sources of the first group 1 there can be solarium tubes and/or UV LED-s, while among light sources of the second group 2 there can be LED-s or even other known devices that emit visible light.

Individual light sources are covered towards the skin surface part B by a separating transparent sheet, which in the case of the second group 2 is a polar filter. At the first group 1 we may apply a special glass or plastic sheet 7 that transmits proper UV radiation. For plastic, mostly polycarbonate (plexi) is used. However, in case of another design, these sheets 7 may have also polarising properties. Such polarising sheet 7 can be produced, for example, that we apply polarising foil onto a transparent glass or plastic sheet, which is also known itself and easily obtainable.

UV light usually is divided in subgroups UV A and UV B according to their wavelength. In case of the present invention we will not enter into details in what subgroups each of these light sources belonging to the second group 2 radiates. Generally we can say that solarium tubes radiate typically in a wide wavelength range, while UV LED devices in a discrete spectrum.

Polarisation in case of the present invention is always linear polarisation. Its favourable physiological effect is known long since. Moreover it is advantageous that as opposed to several other radiation types—for example UV radiation—it can not be overdosed, its overuse has no harmful side effects.

Further it is also possible that light sources of the first group 1, or at least a part of them emits also polarised light towards the skin surface.

To be able to affect on intensity or proportion during operation of light sources belonging to the first 1 and the second 2 groups, expediently a preliminary determination of skin type must be performed. For this we can use advantageously two methods that will be described as follows. In the first method we apply a tabular solution, where the table must be filled so that to provide answers to the included questions as exactly as possible. This method is known formerly in professional practice of determination of skin type and is under continuous development. The questions are directed to obtain various pieces of experimental knowledge to integrate a given person's previous individual experience into the examination. In an expedient case the questions are formulated so that they can be answered by simple decision or with classification into a few number (three, four, etc.) of categories. Based on the answers given to the questions they used to be scored, for example according to an assigned code table, and based on this the scores can be totalised. According to the total number of scores, determination of skin type is possible. An exemplary known table is shown on FIG. 2. Here the type of skin of the person to be examined was classified in four categories. The code table for scoring in this description is not given, its determination can be found in literature in several places, but several slightly different versions can be imagined. Nevertheless, assignment of scores is appears from the figure. Based on the total scores, classification into skin sensitivity categories was performed by distinction of four basic types. These are:

1) very sensible

2) sensible

3) normal

4) resistant to sunlight

Classification can also be performed in such an alternative way that six basic skin types are distinguished. This classification is known from several literature sources. Individual basic skin types in case of the present additional example are as follows:

1) very white

2) white

3) white-light brown

4) light brown

5) dark brown

6) very dark

Of course, other different skin colour and according to this, skin type categories can also be specified, based on what more or less categories can be built.

Score value assignment, as it was previously mentioned, can be performed in several different unique ways, which however, does not reduce effectiveness of the method.

The built categories represent the determinant input control parameter of the device according to the invention. Input of the controlled driver unit of the device is just this data gained in this first step, itself the category determined as a result of skin type determination. This can be provided for the device by manual setting or automatically, if necessary without human intervention, too. In the latter case, for example, the questions of the questionnaire method and the possible answers appear on a screen. Using a proper input device, the answering person selects the correct answers from the screen, and based on them the device can automatically perform the assignment of scores and their summing, then based on the summed scores the classification into the preliminarily determined skin type category.

The second possibility is application of a skin type analyzer unit that is designed to determine the type and properties of a given skin part before treatment. Such devices themselves are also known. Measurement results can be read by an operator and the driver unit of the light therapy skin care device can be controlled and adjusted accordingly. In an alternative way the adjustment can be performed automatically, when the driver unit is controlled by the signal of the skin type analyser unit issued at measurement.

If a skin type analyser unit is used then its expedient way of usage is such that different skin surface parts of the person—whose skin type is to be determined—is lighted and based on the amount and colour of reflected light the skin type, more exactly the skin colour is determined. Among different skin surface parts, expediently at least three different body surface should be selected and measurements should be performed one after the other. These three skin surface parts differ from each other that one of them is continuously subjected to sunshine (e.g. forehead, hand), the second is subjected to average extents (e.g. neck, upper arm), while the third receives the smallest amount of sunshine (e.g. behind knees, armpit). Results received from different skin surface parts are expediently averaged and the result of averaging is taken for the basis of classification into category that constitutes the input of the controlled driver unit.

If necessary, combination of tabular and analyser methods for skin type determination can also be applied. For this, for example the previously described examination series is performed and the result are averaged in weighted way, and based on this the classification is performed. Weighting can be specified according to the fact how realistic results the analyser method provides, which is based only on three measurements, compared to the tabular method where even more than twenty questions can take place. Inaccuracy of the analyser method can cased by the fact that it is suitable for measurement of the momentary status that can also be affected by the condition that the given person is measured before or after sunbathing.

Another possibility is that independently from the way of determination of skin type, with manual or automatic setting, e.g. by selecting from a program library, operation of light sources belonging to the first 1 and to the second 2 groups is controlled by a preliminary adjusted time program. The time program can be an experimental scientific or other time program determined by the manufacturer or by the operator, which controls the operation of individual light source groups with alternating intensity.

To perform intensity control and/or control of intensity rate of light sources of the first 1 and second 2 groups based on the mentioned measurement results or on the output signal of the skin type analyser unit, a proper transformation function is required. This can be control by the time program where the filling factor between the intervals of switch-on and switch-off is changed, or different values are provided in the intensity. In this latter case a suitable light intensity regulation (dimmer) circuit is required. It is to be noted that not every light source is suitable for such regulation.

In an extreme case it can be allowed—knowing the measured condition of the skin—yet the omission of further UV irradiation, or in a reversal case the device can be used only with lighting sources belonging to the first 1 group as a solarium. For this it must be allowed that light emission intensity for one group is zero.

### LIST OF PARTS

B skin surface
1 first group
2 second group
3 solarium tube
4 UV LED
5 LED
6 polar filter
7 sheet

1. A light therapy skin care device including light sources in an arrangement configured for lighting of a given skin surface or the part of the skin surface of human body, where among light sources comprise UV light emitting and UV-free light emitting devices, and the light sources are connected to a controlled driver unit, wherein the light sources are divided in two groups, where in the first group comprise UV light emitting light sources, and in the second group comprise UV-free light emitting light sources, and at least one part of the light sources of the second group emits polarised light towards the skin surface, further the controlled driver unit is adapted to control light emission and/or proportion of light emission from sources of the first and from the second groups.

2. The device according to claim 1, wherein the driver unit is controlled according to a category measured by a skin type analyser unit.

3. The device according to claim 1, wherein the driver unit is controlled according to a category gained on the basis of the survey result gained by filling of the driver unit form.

4. The device according to any one of claims 1, 2 and 3, wherein by the driver unit a time program is selected to control light emission of light sources of the first and second groups or their proportion.

5. The device according to claim 1, wherein polarised light is produced by a polar filter placed between the light source and the skin surface.

6. The device according to claim 1, wherein the controlled driver unit is configured to control light emission intensity of the light sources of the first group, while the light sources of the second group are operated always with maximum power.

7. The device according to claim 1, wherein at least a part of the light sources of the first group emits polarised light towards skin surface.

8. The device according to claim 1, wherein among the light sources of the first group comprise solarium tubes.

9. The device according to claim 1 or 8, wherein among the light sources of the first group comprise UV LED-s.

10. The device according to claim 1, wherein among the light sources of the second group comprise LED-s.

11. Procedure to control the light therapy skin care device according to claim 1, where the given skin surface part of a given person is subjected to determination of 5 skin type, as a result of which a classification into categories is possible, wherein based on this category, light emissions and/or their proportion of the light sources of the first and second group is controlled by the driver unit of the light therapy skin care device.

12. The procedure according to claim 11, wherein skin type determination is performed by a skin type analyser unit.

13. The procedure according to claim 11, wherein skin type determination is performed on the basis of a survey gained by filling of a form.

14. The procedure according to claim 11, wherein proportion of light emission intensity of the light sources of the first and second groups is controlled so that for one group even zero emission is allowed.
